(19) United States
(12) Patent Application Publication
(10) Pub. No.: US 2007/0042938 A1
(43) Pub. Date: Feb. 22, 2007

(54) Use of dpiv and apn inhibitors for the treatment of dermatalogical diseases involving the hyperproliferation and modified differentiation conditions of fibroblasts

(76) Inventors: Siegfried Ansorge, Hohenwarthe (DE); Harald Gollnick, Magdeburg (DE); Uwe Lendeckel, Magdeburg (DE); Klaus Neubert, Halle (DE); Dirk Reinhold, Magdeburg (DE); Robert Vetter, Magdeburg (DE); Burkhart Schraven, Biederitz (DE); Anja Thielitz, Magdeburg (DE); Jurgen Faust, Halle (DE)

Correspondence Address: HODGSON RUSS LLP, ONE M & T PLAZA, SUITE 2000, BUFFALO, NY, 14203-2391, US

(21) Appl. No.: 10/563,498

(22) PCT Filed: Jul. 6, 2004
(86) PCT No.: PCT/EP04/07377

§ 371 Date: Jun. 7, 2006

(30) Foreign Application Priority Data
Jul. 8, 2003 (DE) 103 30 842.3

(51) Int. Cl. A61K 38/04

(52) U.S. Cl. 514/7; 514/19; 514/365; 514/64; 514/419; 514/310

(57) ABSTRACT

The invention relates to a process for the inhibition of the DNA synthesis (essential for the proliferation) of human fibroblasts by a single or joint effect of inhibitors of alanyl aminopeptidase (APN) and dipeptidyl peptidase IV (DPIV) expressed by those cells. The DNA synthesis (proliferation) of human fibroblasts is inhibited, in a dose dependent manner, by an administration of inhibitors of APN or/and DPIV. The invention shows that the application of substances inhibiting the above enzymes or of compositions and administration forms thereof are well suitable for a therapy and a prevention of dermatological diseases associated with fibroblast hyperproliferation and changed fibroblast differentiation states.

The present invention describes the inhibition of fibroblast DNA synthesis essential for the cell proliferation and differentiation by the effect of inhibitors of aminopeptidase N (APN, E.C. 3.4.11.2., CD13) or/and of dipeptidyl peptidase IV (DPIV, E.C. 3.4.14.5., CD26) resulting from the separate, simultaneous or, with respect to time, immediately consecutive application of respective specific inhibitors of those enzymes or of inhibitors of enzymes having an equal substrate specificity (APN- or/and DPIV-analogous enzyme activity) on the basis of amino acid derivatives, peptides or peptide derivatives, by which the proliferation (DNA synthesis) and differentiation of fibroblasts is suppressed and modulated.

A number of dermatologic diseases are accompanied by a hyperproliferation and by changed differentiation states of fibroblasts. Those diseases include benign fibroblast hyperproliferation states (in particular post-infectious, post-inflammatory and post-traumatic: hypertrophic scars, keloids, angiofibroms, dermatofibroms, fibrolipomes, ulcus scars) which may also appear as disseminated (myo-) fibromatoses (for example congenital disseminated fibromatosis), as well as malign fibroblast hyperproliferation states (for example fibrosarcomes, mixed tumors as atypical fibroxanthoma, malign fibrous histiocytoma, aggressive angiomyxoma, paraneoplasiae). Another group of diseases are fibrotic autoimmune diseases as, for example, the localized and systemic sclerodermia in its different occurrences (circumscript sclerodermia, progressive-systemic sclerodermia, CREST syndrome), the dermatosclerosis accompanying other collagenoses and the cutaneous variant of the graft-versus-host disease. Changed differentiation states of the fibroblasts are an expression of a number of fibrotic diseases having, up to now, an etiology which is, to a large extent, still unclear. These diseases include vitiligo (white spot disease, Lichen sclerosus et atrophicus), and the heterogeneous group of pseudosclerodermiae (as, for example the eosinophilic/proliferative fascitis, pseudosclerodermiae generated by exogeneous causes as, for example, toxic oil syndrome, silicosis, porphyriae, eosinophilic myalgia syndrome, popular mucinosis (Lichen myxo-edematosus) or Borrelia-associated fibrosis states). In addition, there are occurring secondary sclerosis conditions as, for example, in the course of a stasis fibrosis accompanying chronic venous insufficiency or lipolymphedemas, in a fibrotic progressive stage of patternal alopecia (alopecia androgenetica) and of rare localized fibroblast diseases (Dupuytren's disease, Ledderhose's disease, “knuckle pads”, penile induration (Peyronie's disease, induratio penis plastica).

Peptidases as, for example, dipeptidyl peptidase IV and aminopeptidase N or ezymes having a similar effect are particularly interesting for the control and modulation of interactions between cells, since they are, in part, localized, as ectoenzymes, in the plasma membrane of the cells, interact with other extracellular structures, activate or inactivate, respectively, peptidergic messenger substances by enzyme-catalyzed hydrolysis and, by that, are important for the cell-cell communication [Yaron A, et al.: Proline-dependent structural and biological properties of peptides and proteins. Crit Rev Biochem Mol Biol 1993;28:31-81; Vanhoof G, et al.: Proline motifs in peptides and their biological processing. FASEB J 1995;9:736-744].

It was shown that membrane-bound peptidases as DP IV and APN play a key role in the process of activation and clonal expansion of immune cells, particularly of T-lymphocytes [Fleischer B: CD26 a surface protease involved in T-cell activation. Immunology Today 1994; 15:180-184; Lendeckel U et al.: Role of alanyl aminopeptidase in growth and function of human T cells. International Journal of Molecular Medicine 1999; 4:17-27; Riemann D et al.: CD13—not just a marker in leukemia typing. Immunology Today 1999; 20: 83-88]. A number of functions of mitogen-stimulated mononuclear cells (MNZ) and enriched T-lymphocytes as, for example, DNA synthesis, production and secretion of immune-stimulating cytokines (IL-2, IL-6, IL-12, IFN-γ) and helper functions of B-cells (synthesis of IgG and IgM) may be inhibited in the presence of specific inhibitors of DP IV or of APN [Schön E et al.: The dipeptidyl peptidase IV, a membrane enzyme involved in the proliferation of T lymphocytes. Biomed. Biochim. Acta 1985; 2: K9-K15; Schön E et al.: The role of dipeptidyl peptidase IV in human T lymphocyte activation. Inhibitors and antibodies against dipeptidyl peptidase IV suppress lymphocyte proliferation and immunoglobulin synthesis in vitro. Eur. J. Immunol. 1987; 17: 1821-1826; Reinhold D et al.: Inhibitors of dipeptidyl peptidase IV induce secretion of transforming growth factor β1 in PWM-stimulated PBMNC and T cells. Immunology 1997; 91: 354-360; Lendeckel U et al.: Induction of the membrane alanyl aminopeptidase gene and surface expression in human T-cells by mitogenic activation. Biochem. J. 1996; 319: 817-823; Kähne T et al.: Dipeptidyl peptidase IV: A cell surface peptidase involved in regulating T cell growth (Review). Int. J. Mol. Med. 1999; 4: 3-15; Lendeckel U et al.: Role of alanyl aminopeptidase in growth and function of human T cells (Review). Int. J. Mol. Med. 1999; 4: 17-27].

It is already known that the treatment of autoimmune diseases and graft-versus-host rejections may be achieved by an inhibition of dipeptidyl peptidase IV localized on immune cells by means of synthetic inhibitors (see, for example, EP-A 0 764 151; WO 95/29691, EP-A 0 731 789, EP-A 0 528 858).

The present invention is based on the surprising finding that the isolated or simultaneous effect of inhibitors of dipeptidyl peptidase IV/DP IV or CD 26 (expressed on or in fibroblasts) or of inhibitors of enzymes having an equal substrate specificity (DP IV-analogous enzyme activity) and of inhibitors of aminopeptidase N/APN or CD13 or of inhibitors of enzymes having an equal substrate specificity (APN-analogous enzyme activity) inhibits the proliferation (DNA synthesis) of fibroblasts.

The invention shows that the single or simultaneous application of substances inhibiting DP IV and APN or of substances inhibiting enzymes having an equal substrate specificity (APN- or/and DP IV-analogous enzyme activity) or the application of corresponding compositions and administration forms thereof are well suitable for a therapy and a prevention of dermatological diseases associated with fibroblast hyperproliferation and changed differentiation states, for whose development the proliferation and the differentiated control of the DNA synthesis of fibroblasts plays a central role.

In detail, the invention is based on the findings that the DNA synthesis of fibroblasts is significantly inhibited by the administration of inhibitors of dipeptidyl peptidase IV or of inhibitors of enzymes having an equal substrate specificity or/and of inhibitors of aminopeptidase N or of inhibitors of enzymes having an equal substrate specificity.

The above-mentioned diseases are presently treated topically and/or systemically by an administration of immunosuppressive agents, glucocorticosteroids, unspecific anti-inflammatory agents and emollients, and are treated symptomatically by physiotherapy. Particularly in connection with the systemic medication, often undesired side effects are occurring, amongst others the cushing syndrome, osteoporosis, infections or diabetes mellitus. In connection with a topical therapy, local skin atropiae and an increased skin vulnerability are prevailing. In connection with the systemic therapy, and with the topical immunosuppressive therapy as well, skin tumors may be propagated.

When considering the above-mentioned diseases, and in particular their early stages, the use of inhibitors of DP IV or/and of APN would be a completely new, presumably very effective, possibly low-cost form of a therapy and a valuable alternative component of existing therapy concepts.

The inhibitors, applied in accordance with the invention, of dipeptidyl peptidase IV or the inhibitors of enzymes having an equal substrate specificity (DP IV-analogous enzyme activity) or/and the inhibitors of aminopeptidase N or the inhibitors of enzymes having an equal substrate specificity (APN-analogous enzyme activity) may be applied in pharmaceutically applicable formulation complexes as inhibitors, substrates, pseudosubstrates, peptides and peptide derivatives acting as inhibitor(s) and as antibodies of such enzymes as well. The inhibitors of the invention are employed alone or as a combination of several of them, preferably as a combination of two of them.

Preferred effectors for DP IV are: Xaa-Pro dipeptides, corresponding derivatives, preferably dipeptide phosphonic acid diaryl esters, dipeptide boronic acids (for example Pro-Boro-Pro) and their salts, Xaa-Xaa-(Trp)-Pro-(Xaa)ₙ peptides (n=0 to 10), corresponding derivatives and their salts, and amino acid-(Xaa) amides, corresponding derivatives and their salts, wherein Xaa represents an α-amino acid/-imino acid or an α-amino acid derivative/-imino acid derivative, preferably N^{ε}-4-nitrobenzyloxycarbonyl-L-lysine, L-proline, L-tryptophane, L-iso-leucine, L-valine, and cyclic amines, for example pyrrolidine, piperidine, thiazolidine and their derivatives represent the amide structure. Such compounds and their preparation were desribed in an earlier patent (K. Neubert et al., DD 296,075 A5).

Furthermore, tryptophane-1,2,3,4-tetrahydroisoquinoline-3-carboxylic acid derivatives (TSL) and (2S, 2S′, 2S″)-2-[2′-[2″-amino-3″-(indole-3′″-yl-) 1″-oxoprol-yl-]1′,2′,3′,4′-tetrahydro-6′,8′-dihydroxy-7-methoxy-isoquinol-3-yl-carbonylamino-]4-hydroxymethyl-5-hydro-pentanoic acid (TMC-2A) may be used as effectors for DP IV advantageously. An exemplary DP IV inhibitor which may be used advantageously, is Lys[Z(NO₂)] thiazolidide, wherein Lys represents a L-lysine residue, and Z(NO₂) represents 4-nitrobenzyloxycarbonyl (see also DD-A 296,075).

Considered as exemplary inhibitors of alanyl aminopeptidase (aminopeptidase N, APN) are, in accordance with the invention: actinonin, leuhistin, phebestin, amastatin, bestatin, probestin, β-amino thiols, α-amino phosphinic acids, α-amino phosphinic acid derivatives, preferably D-Phe-ψ-[PO(OH)—CH₂]-Phe-Phe and their salts. Preferred inhibitors for alanyl aminopeptidase are bestatin (Ubenimex), actinonin, probestin, phebestin, RB3014 or leuhistin.

The inhibitors and pharmaceutical preparations containing them are administered simultaneously together with known carrier substances. Comprised by the invention are also pharmaceutical preparations which comprise two or more of the inhibitors of DP IV or of the inhibitors of enzymes having a DP IV-analogous enzyme activity or/and of the inhibitors of APN or of the inhibitors of enzymes having an APN-analogous enzyme activity and are in a spaced apart formulation in combination with per se known carrier, auxiliary and/or additive substances for a simultaneous or, with respect to time, immediately consecutive administration with the aim of a joint effect.

The administration is made, on the one hand, as a topical application in the form of, for example, creams, ointments, pastes, gels, solutions, sprays, liposomes and nanosomes, mixable lotions, “pegylated” formulations, degradable (i.e. decomposable under physiological conditions) depot matrices, hydrocolloid dressings, plasters, microsponges, prepolymers and similar new carrier substrates, jet injections and other dermatological bases/vehicles including instillative application; and, on the other hand, as a systemic application for an oral, transdermal, intravenous, subcutaneous, intracutaneous, intramuscular application in suitable recipes or in a suitable galenic form.

The inhibitor(s) according to the invention as well as preparations containing one or several of the above-mentioned inhibitors and, optionally, further components as, for example, further inhibitors and pharmaceutically acceptable additive, auxiliary or carrier substances may be applied preventively or therapeutically in a multiplicity of dermatological diseases and conditions including a hyperproliferation and changed differentiation states of fibroblasts. As examples, there may be mentioned a prevention and therapy of benign fibrotic and sclerotic diseases (in particular post-infectious and post-traumatic: hypertrophic scars, keloids, dermatofibroms, fibrolipomes, disseminated (myo-) fibromatoses), as well as of malign fibroblast hyperproliferation states (for example fibrosarcomes, mixed tumors as atypical fibroxanthoma, malign fibrous histiocytoma, aggressive angiomyxoma, paraneoplasiae), of fibrotic autoimmune diseases as, for example, sclerodermia (circumscript sclerodermia, progressive-systemic sclerodermia, CREST syndrome), of dermatosclerosis accompanying other collagenoses and the graft-versus-host disease, of vitiligo (white spot disease, Lichen sclerosus et atrophicus), and of the heterogeneous group of pseudosclerodermiae (as, for example the eosinophilic/proliferative fascitis, pseudosclerodermiae generated by exogeneous causes as, for example, toxic oil syndrome, silicosis, porphyriae, eosinophilic myalgia syndrome, popular mucinosis (Lichen myxo-edematosus) or Borrelia-associated fibrosis states), of secondary sclerosis conditions as, for example, in the course of a stasis fibrosis accompanying chronic venous insufficiency or lipolymphedemas, in a fibrotic progressive stage of patternal alopecia (alopecia androgenetica) and of rare localized fibro-blast diseases (Dupuytren's disease, Ledderhose's disease, “knuckle pads”, penile induration (Peyronie's disease, induratio penis plastica).

The invention also relates to a process for the therapy and prevention of dermatologic diseases including a hyperproliferation and changed differentiation states of fibroblasts, which process comprises an administration of inhibitors of dipeptidyl peptidase IV (DP IV) as well as of inhibitors of enzymes having an equal substrate specificity (DP IV-analogous enzyme activity) or/and of inhibitors of alanyl aminopeptidase (aminopeptidase N, APN) as well as of inhibitors of enzymes having an equal substrate specificity (APN-analogous enzyme activity) to a patient in need of a treatment for a prevention and/or therapy of the above-mentioned dermatological diseases.

In a particularly preferred embodiment of the invention, one inhibitor or several inhibitors of the above-mentioned enzymes, or one or several pharmaceutical preparation(s) containing these inhibitors singly or—preferably—in combination are administered to a patient suffering from one or several of the diseases mentioned subsequently or needing a prevention against any of the diseases mentioned subsequently. Said inhibitors are preferably selected from inhibitors of DP IV and, particularly preferably, from Xaa-Pro-dipeptides (Xaa=α-amino acid or side chain-protected derivative) corresponding derivatives, more preferably dipeptide phosphonic acid diaryl esters, dipeptide boronic acids (e.g. Pro-Boro-Pro) and their salts, Xaa-Xaa-(Trp)-Pro-(Xaa)ₙ peptides (Xaa=α-amino acid, n=0 to 10), corresponding derivatives and their salts, amino acid (Xaa) amides, corresponding derivatives and their salts, wherein Xaa represents an α-amino acid or a side chain-protected derivative, preferably N-4-nitrobenzyl-oxycarbonyl-L-lysine, L-proline, L-tryptophane, L-isoleucine, L-valine, and cyclic amines, for example pyrrolidine, piperidine, thiazolidine and their derivatives, represent the amide structure, and/or tryptophane-1,2,3,4-tetrahydroisoquinoline-3-carboxylic acid derivatives (TSL) and (2S 2S′, 2S″)-2-[2′-[2″-amino-3″-(indole-3′″-yl-) 1″-oxoprolyl-]1′,2′,3′,4′-tetrahydro-6′,8′-dihydroxy-7-methoxy-isoquinol-3-yl-carbonylamino-]4-hydroxymethyl-5-hydro-pentanoic acid (TMC-2A); and from inhibitors of APN and, particularly preferably, actinonin, leuhistin, phebestin, amastatin, bestatin, probestin, β-amino thiols, α-amino phosphinic acids, α-amino phosphinic acid derivatives, preferably D-Phe-ψ-[PO(OH)—CH₂]-Phe-Phe and their salts.

In further preferred methods of the invention, the inhibitors and, optionally, their combinations with each other and pharmaceutical preparations containing those inhibitors or combinations thereof are applied for a prevention and therapy of diseases and conditions including a hyperproliferation and changed differentiation states of fibroblasts. As examples, there may be mentioned: a prevention and therapy of benign fibrotic and sclerotic diseases (in particular post-infectious and post-traumatic: hypertrophic scars, keloids, dermatofibroms, fibrolipomes, disseminated (myo-) fibromatoses), as well as of malign fibroblast hyperproliferation states (for example fibrosarcomes, mixed tumors as atypical fibroxanthoma, malign fibrous histiocytoma, aggressive angiomyxoma, paraneoplasiae), of fibrotic autoimmune diseases as, for example, sclerodermia (circumscript sclerodermia, progressive-systemic sclerodermia, CREST syndrome), of dermatosclerosis accompanying other collagenoses and the graft-versus-host disease, of vitiligo (white spot disease, Lichen sclerosus et atrophicus), and of the heterogeneous group of pseudosclerodermiae (as, for example the eosinophilic/proliferative fascitis, pseudosclerodermiae generated by exogeneous causes as, for example, toxic oil syndrome, silicosis, porphyriae, eosinophilic myalgia syndrome, popular mucinosis (Lichen myxoedematosus) or Borrelia-associated fibrosis states), of secondary sclerosis conditions as, for example, in the course of a stasis fibrosis accompanying chronic venous insufficiency and lipolymphedemas, in a fibrotic progressive stage of patternal alopecia (alopecia androgenetica) and of rare localized fibroblast diseases (Dupuytren's disease, Ledderhose's disease, “knuckle pads”, penile induration (Peyronie's disease, induratio penis plastica).

In prevention and/or therapy methods particularly preferred in accordance with the invention, one or several of the inhibitors of DP IV and/or APN mentioned above are applied in such a way that two or more of the inhibitors of DP IV or of inhibitors of enzymes having a DP IV-analogous enzyme activity or/and of inhibitors of APN or of inhibitors of enzymes having an APN-analogous enzyme activity are administered in the form of spaced-apart, separate formulations in combination with per se known carrier, auxiliary and/or additive substances simultaneously or, with respect to the time, immediately consecutive with the aim of a joint effect. The administration is in the form of a systemic application for oral, transdermal, percutaneous, intravenous, subcutaneous, intracutaneous, intramuscular, rectal, vaginal, sublingual application together with per se known carrier, auxiliary and/or additive substances and/or as a topical application in the form of creams, ointments, pastes, gels, solutions, sprays, liposomes or nanosomes, “pegylated” formulations, degradable depot matrices, mixable lotions, hydrocolloid dressings, plasters, microsponges, prepolymers and similar new carrier substrates, jet injections and other dermatological bases/vehicles including instillative application.

The invention is in more detail explained by means of the following examples. The examples show preferred embodiments of the invention. However, the invention is not restricted to the preferred embodiments.

### EXAMPLES

### Working Example 1

Our investigations show that the DNA synthesis of human fibroblasts is inhibited by an administration of inhibitors of DP IV (Lys[Z(NO₂)] thiazolidide) or/and of APN (Actinonin) in a dose-dependent manner.

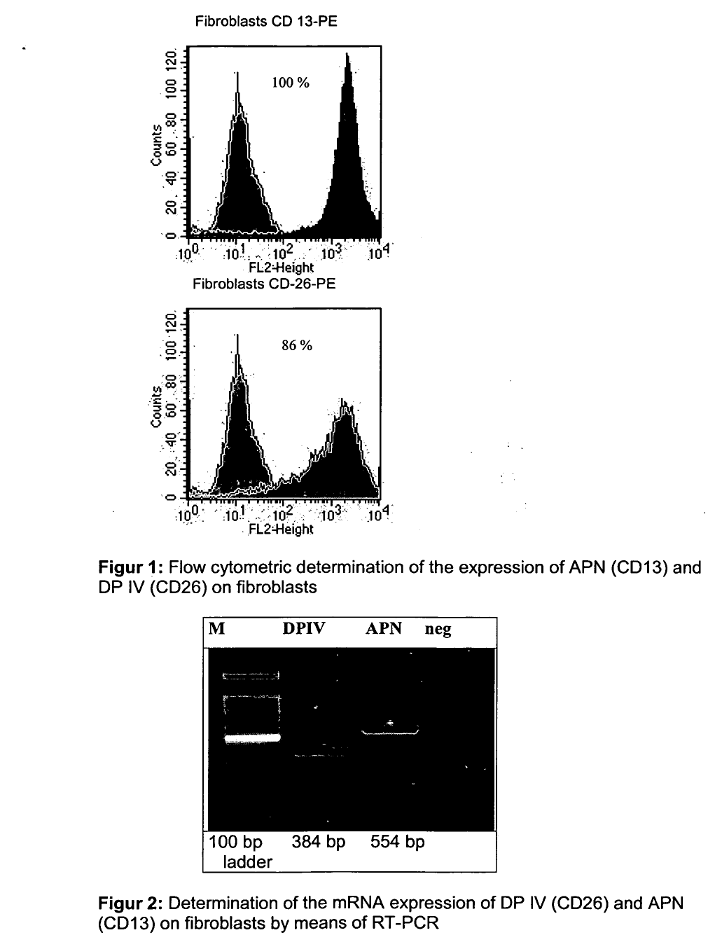

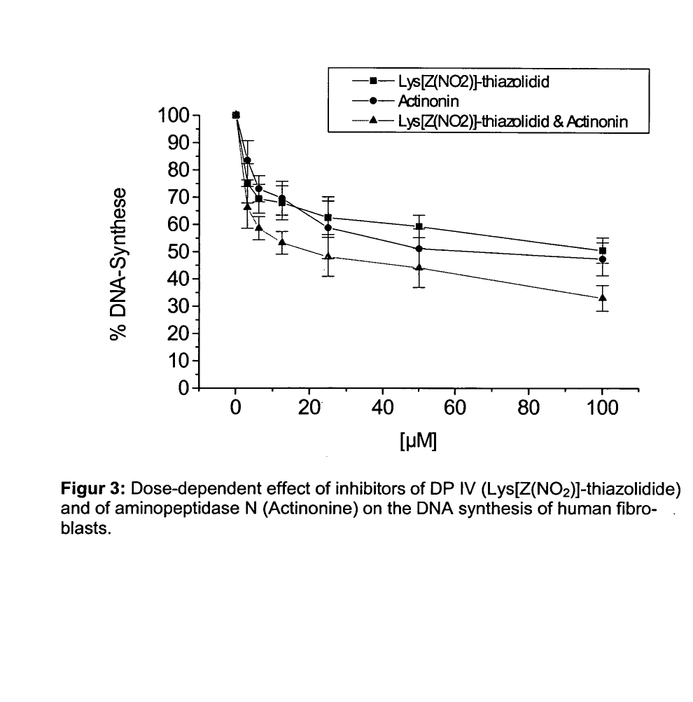

Human fibroblasts strongly express DP IV and APN (FIG. 1). The enzyme activity of DP IV in vital cells is 57.3±12.4 pkat/10⁶ cells, and the enzyme activity of APN is 380.5±48.2 pkat/10⁶ cells (n=4). Correspondingly, the mRNA of APN and DP IV can be detected on such cells (FIG. 2).

Fibroblasts of healthy donors were incubated for 48 h together with the above-mentioned inhibitors, and the DNA synthesis was determined subsequently by a measurement of the ³[H]-thymidine incorporation, as described by Reihold et al. (Reinhold D et al.: Inhibitors of dipeptidyl peptidase IV induce secretion of transforming growth factor β1 in PWM-stimulated PBMNC and T cells, Immunology 1997, 91: 354-360). FIG. 3 shows the dose-dependent inhibition of the DNA synthesis.

For measuring the dose-dependent effect of inhibitors of DP IV (Lys[Z(NO₂)] thiazolidide) and of aminopeptidase N (Actinonin) on the DNA synthesis of human fibroblasts, cells were incubated for 48 h with the indicated concentrations of the inhibitors. Subsequently, ³[H]-Methyl thymidine was added to the culture medium, and the amount of ³[H]-thymidine incorporated into the DNA was measured after further 6 h. The results are shown in FIG. 3.

1. A method for inhibiting proliferation (DNA synthesis) of human fibroblasts comprising utilizing inhibitors of dipeptidyl peptidase IV (DP IV) as well as of inhibitors of enzymes having an equal substrate specificity (DP IV-analogous enzyme activity) or/and of inhibitors of alanyl aminopeptidase (aminopeptidase N, APN) as well as of inhibitors of enzymes having an equal substrate specificity (APN-analogous enzyme activity) for an inhibition of the proliferation (DNA synthesis) of human fibroblasts.

2. The method according to claim 1, wherein the DP IV inhibitors are Xaa-Pro-dipeptides (Xaa=α-amino acid or side chain-protected derivative), corresponding derivatives, more preferably dipeptide phosphonic acid diaryl esters, dipeptide boronic acids (e.g. Pro-Boro-Pro) and their salts, Xaa-Xaa-(Trp)-Pro-(Xaa)ₙ peptides (Xaa=α-amino acid, n=0 to 10), corresponding derivatives and their salts, amino acid (Xaa) amides, corresponding derivatives and their salts, wherein Xaa represents an α-amino acid or a side chain-protected derivative, preferably N^{ε}-4-nitrobenzyl-oxycarbonyl-L-lysine, L-proline, L-tryptophane, L-isoleucine, L-valine, and cyclic amines, for example pyrrolidine, piperidine, thiazolidine and their derivatives, represent the amide structure, and/or tryptophane-1,2,3,4-tetrahydroisoquinoline-3-carboxylic acid derivatives (TSL) and (2S, 2S′, 2S″)-2-[2′-[2″-amino-3″-(indole-3′″-yl-) 1″-oxoprolyl-]1′,2′,3′,4′-tetrahydro-6′,8′-dihydroxy-7-methoxy-isoquinol-3-yl-carbonylamino-] 4-hydroxymethyl-5-hydro-pentanoic acid (TMC-2A).

3. The method according to claim 1, wherein amino acid amides, preferably N^{ε}-4-nitrobenzyloxycarbonyl-L-lysine thiazolidide, pyrrolidide and piperidide as well as the corresponding 2-cyanothiazolidide, 2-cyanopyrrolidide and 2-cyanopiperidide derivative are used as the DP IV inhibitors.

4. The method according to claim 1, wherein the inhibitors of APN are actinonin, leuhistin, phebestin, amastatin, bestatin, probestin, β-amino thiols, α-amino phosphinic acids, α-amino phosphinic acid derivatives, preferably D-Phe-ψ-[PO(OH)—CH₂]-Phe-Phe and their salts.

5. A method utilizing the inhibitor combinations according to claim 1 for a prevention and therapy of benign fibrotic and sclerotic diseases (in particular post-infectious and post-traumatic: hypertrophic scars, keloids, dermatofibroms, fibrolipomes, disseminated
(myo-) fibromatoses), as well as of malign fibroblast hyperproliferation states (for example fibrosarcomes, mixed tumors as atypical fibroxanthoma, malign fibrous histiocytoma, aggressive angiomyxoma, paraneoplasiae), of fibrotic autoimmune diseases as, for example, sclerodermia (circumscript sclerodermia, progressive-systemic sclerodermia, CREST syndrome), of dermatosclerosis accompanying other collagenoses and the graft-versus-host disease, of vitiligo (white spot disease, Lichen sclerosus et atrophicus), and of the heterogeneous group of pseudosclerodermiae (as, for example the eosinophilic/proliferative fascitis, pseudosclerodermiae generated by exogeneous causes as, for example, toxic oil syndrome, silicosis, porphyriae, eosinophilic myalgia syndrome, popular mucinosis (Lichen myxo-edematosus) or Borrelia-associated fibrosis states), of secondary sclerosis conditions as, for example, in the course of a stasis fibrosis accompanying chronic venous insufficiency or lipolymphedemas, in a fibrotic progressive stage of patternal alopecia (alopecia androgenetica) and of rare localized fibroblast diseases (Dupuytren's disease, Ledderhose's disease, “knuckle pads”, penile induration (Peyronie's disease, induratio penis plastica).

6. Pharmaceutical preparations, comprising inhibitors of dipeptidyl peptidase IV (DP IV) as well as inhibitors of enzymes having DP IV-analogous enzyme activity or/and inhibitors of alanyl aminopeptidase (aminopeptidase N, APN) as well as inhibitors of enzymes having APN-analogous enzyme activity, in combination with per se known carrier, additive and/or auxiliary substances.

7. Pharmaceutical preparations according to claim 6, comprising, as the DP IV inhibitors, Xaa-Pro-dipeptides (Xaa=α-amino acid or side chain-protected derivatives), corresponding derivatives, more preferably dipeptide phosphonic acid diaryl esters, dipeptide boronic acids (e.g. Pro-Boro-Pro) and their salts, Xaa-Xaa-(Trp)-Pro-(Xaa)ₙ peptides (Xaa=α-amino acid, n=0 to 10), corresponding derivatives and their salts, amino acid (Xaa) amides, corresponding derivatives and their salts, wherein Xaa represents an α-amino acid or a side chain-protected derivative, preferably N^{ε}-4-nitrobenzyloxycarbonyl-L-lysine, L-proline, L-tryptophane, L-isoleucine, L-valine, and cyclic amines, for example pyrrolidine, piperidine, thiazolidine and their derivatives, represent the amide structure.

8. Pharmaceutical preparations according to claim 6, comprising, as the DP IV inhibitors, preferably amino acid amides, for example N-4-nitrobenzyl-oxycarbonyl-L-lysine thiazolidide, pyrrolidide and piperidide as well as the corresponding 2-cyanothiazolidide, 2-cyanopyrrolidide and 2-cyanopiperidide derivative.

9. Pharmaceutical preparations according to claim 6, comprising, as the APN inhibitors, actinonin, leuhistin, phebestin, amastatin, bestatin, probestin, β-amino thiols, α-amino phosphinic acids, α-amino phosphinic acid derivatives, preferably D-Phe-ψ-[PO (OH)—CH₂] -Phe-Phe and their salts.

10. Pharmaceutical preparations according to claim 6, comprising two or several inhibitors of DPIV or inhibitors of enzymes having a DPIV-analogous enzyme activity or/and inhibitors of APN or inhibitors having an APN-analogous enzyme activity in a spacely separated formulation in combination with per se known carrier, auxiliary and/or additive substances for a simultaneous or, with respect to time, immediately consecutive administration with the aim of a joint effect.

11. Pharmaceutical preparations according to claim 6 for a systemic application for an oral, transdermal, percutaneous, intravenous, subcutaneous, intracutaneous, intramuscular, rectal, vaginal, sublingual application together with per se known carrier, auxiliary and/or additive substances.

12. Pharmaceutical preparations according to claim 6 for a topical application in the form of creams, ointments, pastes, gels, solutions, sprays, liposomes or nanosomes, “pegylated” formulations, degradable depot matrices, mixable lotions, hydrocolloid dressings, plasters, microsponges, prepolymers or other dermatological bases/vehicles including instillative application.

13. A method for the therapy and prevention of dermatological diseases including a hyperproliferation and changed differentiation states of fibroblasts, comprising the administration of inhibitors of dipeptidyl peptidase IV (DPIV) as well as of inhibitors of enzymes having an equal substrate specificity (DPIV-analogous enzyme activity) or/and of inhibitors of alanyl aminopeptidase (aminopeptidase N, APN) as well as of inhibitors of enzymes having an equal substrate specificity (APN-analogous enzyme activity).

14. The method of claim 13, wherein one inhibitor or several inhibitors of the above enzymes or one or several preparation(s) containing those inhibitors singly or—preferably—in combination is/are administered to a patient, said inhibitors being selected from inhibitors of DPIV and particularly preferable from Xaa-Pro-dipeptides (Xaa=α-amino acid or side chain-protected derivative), corresponding derivatives, more preferably dipeptide phosphonic acid diaryl esters, dipeptide boronic acids (e.g. Pro-Boro-Pro) and their salts, Xaa-Xaa-(Trp)-Pro-(Xaa)ₙ peptides (Xaa=α-amino acid, n=0 to 10), corresponding derivatives and their salts, amino acid (Xaa) amides, corresponding derivatives and their salts, wherein Xaa represents an α-amino acid or a side chain-protected derivative, preferably N^{ε}-4-nitrobenzyloxycarbonyl-L-lysine, L-proline, L-tryptophane, L-isoleucine, L-valine, and cyclic amines, for example pyrrolidine, piperidine, thiazolidine and their derivatives, represent the amide structure, and/or tryptophane-1,2,3,4-tetrahydroisoquinoline-3-carboxylic acid derivatives (TSL) and (2S, 2S′, 2S″)-2-[2′-[2″-amino-3″-(indole-3′″-yl-) 1″-oxoprolyl-]1′,2′,3′,4′-tetrahydro-6′,8′-dihydroxy-7-methoxyiso-quinol-3-yl-carbonylamino-] 4-hydroxymethyl-5-hydro-pentanoic acid (TMC-2A); and from inhibitors of APN and particularly preferable from actinonin, leuhistin, phebestin, amastatin, bestatin, probestin, β-amino thiols, α-amino phosphinic acids, α-amino phosphinic acid derivatives, preferably D-Phe-ψ-[PO(OH)—CH₂]-Phe-Phe and their salts.

15. Pharmaceutical preparations according to claim 7 comprising two or several inhibitors of DPIV or inhibitors of enzymes having a DPIV-analogous enzyme activity or/and inhibitors of APN or inhibitors having an APN-analogous enzyme activity in a spacely separated formulation in combination with per se known carrier, auxiliary and/or additive substances for a simultaneous or, with respect to time, immediately consecutive administration with the aim of a joint effect.

16. Pharmaceutical preparations according to claim 8, comprising two or several inhibitors of DPIV or inhibitors of enzymes having a DPIV-analogous enzyme activity or/and inhibitors of APN or inhibitors having an APN-analogous enzyme activity in a spacely separated formulation in combination with per se known carrier, auxiliary and/or additive substances for a simultaneous or, with respect to time, immediately consecutive administration with the aim of a joint effect.

17. Pharmaceutical preparations according to claim 9, comprising two or several inhibitors of DPIV or inhibitors of enzymes having a DPIV-analogous enzyme activity or/and inhibitors of APN or inhibitors having an APN-analogous enzyme activity in a spacely separated formulation in combination with per se known carrier, auxiliary and/or additive substances for a simultaneous or, with respect to time, immediately consecutive administration with the aim of a joint effect.
